(19) United States
(12) Patent Application Publication
(10) Pub. No.: US 2018/0199701 A1
(43) Pub. Date: Jul. 19, 2018

(54) COSMETIC BRUSH HEAD INCLUDING ANTI-AGING INGREDIENTS

(71) Applicant: L'Oreal USA Creative, Inc., New York, NY (US)

(72) Inventors: Jamie Kern, Jersey City, NJ (US); Regina Baptista, Jersey City, NJ (US)

(21) Appl. No.: 15/871,610

(22) Filed: Jan. 15, 2018

Related U.S. Application Data
(60) Provisional application No. 62/446,596, filed on Jan. 16, 2017.

(51) Int. Cl. A46B 11/00; A46B 9/02; A46D 1/00; A61Q 19/08

(52) U.S. Cl. CPC A46B 11/0072; A46B 9/021; A46D 1/0207; A61K 8/65; A46B 2200/1046; A61K 2800/87; A61Q 19/08

(57) ABSTRACT

The present disclosure provides an applicator for applying cosmetics, the applicator having anti-aging ingredients infused therein. The applicator includes a tuft of anti-aging filaments including at least one anti-aging material and synthetic grain. The applicator also includes an applicator body physically coupled to the tuft of anti-aging filaments.

### RELATED APPLICATIONS

This application claims the benefit of Application No. 62/446,596 filed on Jan. 16, 2017, which is herein incorporated by reference in its entirety.

### FIELD OF INVENTION

The present disclosure relates generally to the field of cosmetic brushes and more particularly to a cosmetic brush head having anti-aging ingredients infused therein.

### BACKGROUND

The cosmetic industry sells numerous products directed to slowing or erasing the effects of age on one's appearance (such as wrinkles, age spots, or loss of skin elasticity, etc.). For this reason, many efforts have been made to create anti-aging products that nourish, repair, or supplement one's skin so as to create a more youthful appearance. These anti-aging products typically take the form of lotions, serums, or powders that a user applies separately, e.g., before and in addition to their normal cosmetic routine. This requires a user to spend additional time applying the anti-aging products when using cosmetics that do not already include anti-aging ingredients.

### SUMMARY OF INVENTION

The present disclosure provides a method of making a cosmetic brush head, having anti-aging ingredients infused therein. The resulting brush head obviates the need for a user to apply anti-aging ingredients separately when applying cosmetics that do not include anti-aging ingredients, as the brush head simultaneously administers both anti-aging ingredients as well as the other cosmetic product being applied with the brush head. The anti-aging ingredients are infused within the brush head hairs themselves and transfer anti-aging ingredients to the user's skin upon contact

According to one aspect, there is provided an applicator for applying a cosmetic composition. The applicator includes a tuft of anti-aging filaments including at least one anti-aging material and at least one of polybutylene terephthalate (PBT), polyethylene terephthalate (PET), nylon, polyester, a thermoplastic elastomer (TPE), or combinations thereof. The applicator also includes an applicator body physically coupled to the tuft of anti-aging filaments.

Alternatively or additionally, at least one anti-aging material includes at least one of vitamin A, vitamin C, vitamin E, hydrolyzed collagen, niacin, peptides, antioxidants, and hydrolyzed silk.

Alternatively or additionally, at least one anti-aging material includes at least one of collagen peptides, elastin, peptides, copper peptides.

Alternatively or additionally, at least one anti-aging material includes at least one of hexapeptides, palmitoyl oligopeptide, palmitoyl Tetrapeptide-7, and pentapeptides.

Alternatively or additionally, a length of the tuft of anti-aging filaments comprises between 15% and 20% of a length of the applicator.

Alternatively or additionally, a width at a widest of the tuft of anti-aging filaments is between 50% and 60% wider than a width at a widest point of the applicator body.

Alternatively or additionally, a width of the applicator body at a widest point is between 60% and 70% wider than a width of the applicator body at a narrowest point.

Alternatively or additionally, a width of the tuft of anti-aging filaments at a widest point is between 50% and 60% wider than a width of the tuft of anti-aging filaments at a narrowest point.

Alternatively or additionally, a shape of the tuft of anti-aging filaments is configured to conform to a curve of a face of a user when the tuft of anti-aging filaments is brushed against the face of the user.

Alternatively or additionally, an angle of a top surface of the tuft of anti-aging filaments located opposite the applicator body is configured to conform to the curve of the face of the user when the tuft of anti-aging filaments is brushed against the face of the user.

Alternatively or additionally, the shape of the tuft of anti-aging filaments is configured to conform to a cheek of the face of the user when the tuft of anti-aging filaments is brushed against the cheek of the user.

According to another aspect, there is provided a cosmetic styling tool including a brush head having a plurality of filaments including an admixture of one or more anti-aging materials and one or more polymer materials. The cosmetic styling tool also includes a styling tool handle.

Alternatively or additionally, the plurality of filaments comprises an admixture of one or more antioxidants and one or more polymer materials.

Alternatively or additionally, the plurality of filaments comprises an admixture of anti-aging material and at least one of polybutylene terephthalate (PBT), polyethylene terephthalate (PET), nylon, polyester, a thermoplastic elastomer (TPE), or combinations thereof.

Alternatively or additionally, the plurality of filaments comprises an admixture of one or more of vitamin A, vitamin C, vitamin E, hydrolyzed collagen, niacin, peptides, antioxidants, or hydrolyzed silk, or combinations thereof, and one or more polymer materials.

Alternatively or additionally, the plurality of filaments comprises an admixture of one or more of collagen peptides, elastin, peptides, or copper peptides, and one or more polymer materials.

Alternatively or additionally, the plurality of filaments comprises an admixture of one or more of hexapeptides, palmitoyl oligopeptide, palmitoyl Tetrapeptide-7, and pentapeptides, and one or more polymer materials.

Alternatively or additionally, the brush head comprises a cross-section having a regular or an irregular geometric shape.

The foregoing and other features of the invention are hereinafter fully described and particularly pointed out in the claims, the following description and annexed drawings setting forth in detail certain illustrative embodiments of the invention, these embodiments being indicative, however, of but a few of the various ways in which the principles of the invention may be employed.

Features that are described and/or illustrated with respect to one embodiment may be used in the same way or in a similar way in one or more other embodiments and/or in combination with or instead of the features of the other embodiments.

### BRIEF DESCRIPTION OF THE DRAWINGS

FIG. 1 is a flowchart illustrating the ordered steps of a method for manufacturing a brush head including anti-aging ingredients.

FIG. 2 is a flowchart illustrating the ordered steps of the method of FIG. 1 within the context of machines used during the method.

FIGS. 3A-3E shows exemplary embodiments of cosmetic brush heads having various shapes.

### DETAILED DESCRIPTION OF THE DRAWINGS

The present disclosure provides an anti-aging cosmetic brush head and a method for making the anti-aging cosmetic brush head.

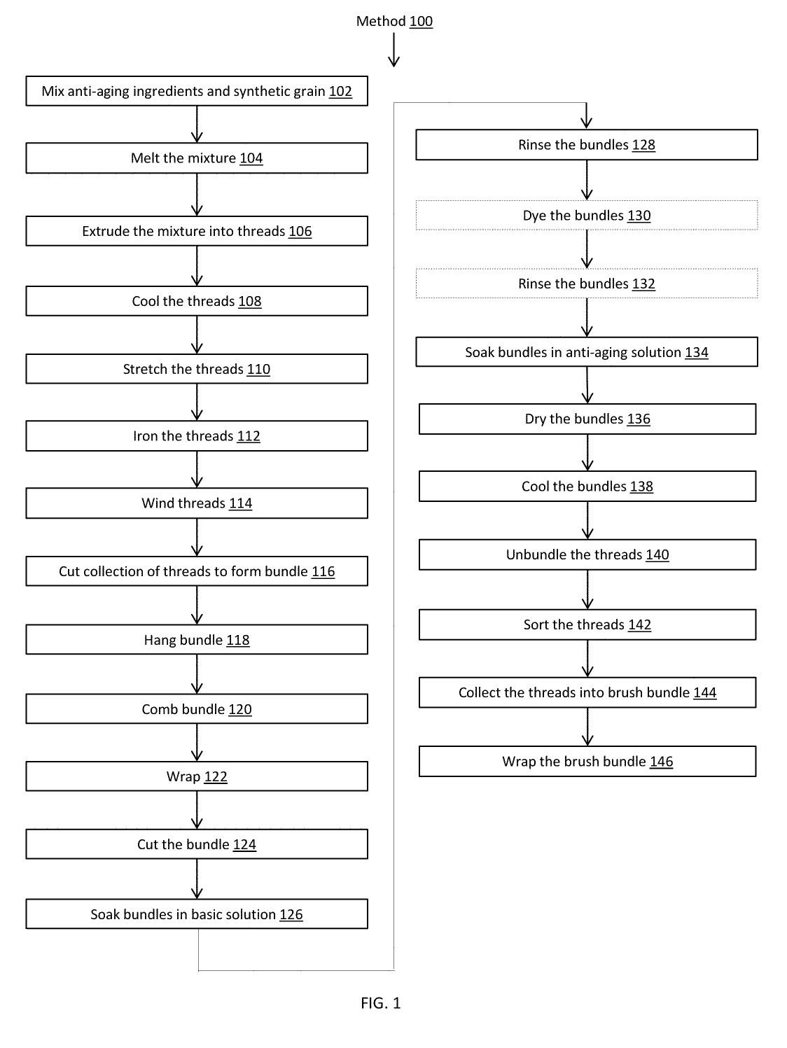

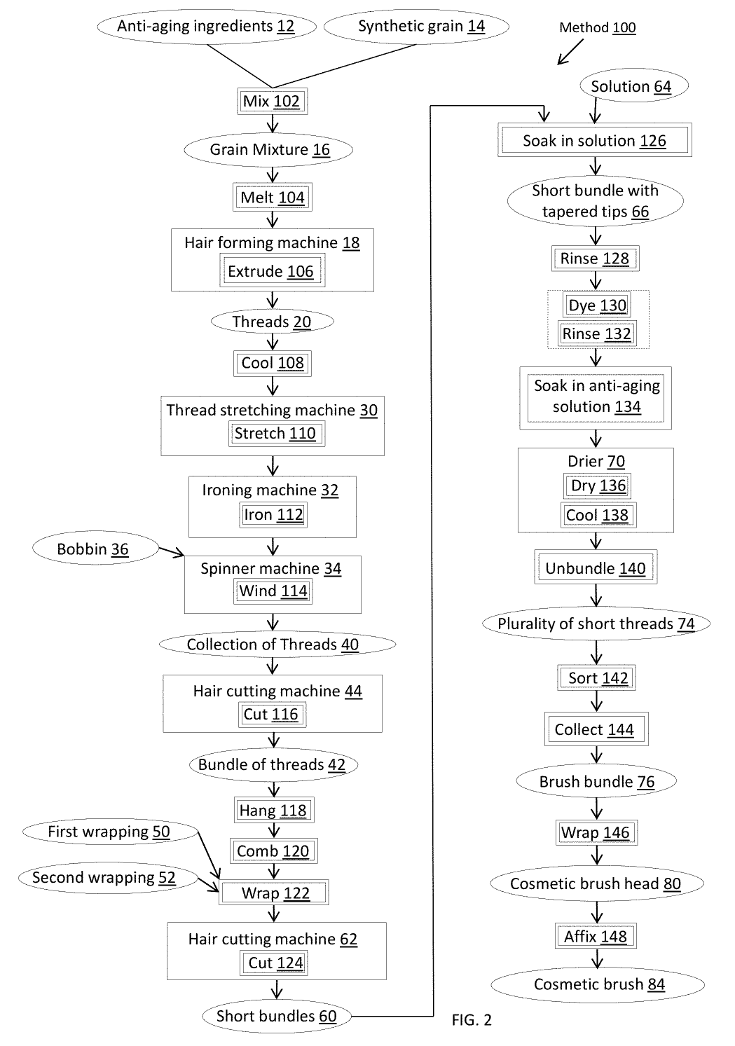

Turning to FIG. 1, the steps of a method 100 of making an anti-aging cosmetic brush head 80 are shown in order. FIG. 1 shows the order of steps, with optional steps indicated by dotted borders. FIG. 2 shows the same method 100 within the context of machines used. The steps of the method 100 in FIG. 2 are indicated by a double border, while any devices used to perform the steps are indicated by a single border. Further, any intermediary product or ingredient resulting or used in the method 100 is indicated by an oval.

Turning to FIG. 2, a method 100 of making an anti-aging cosmetic brush head 80 is described. In process block 102, anti-aging ingredients 12 and synthetic grain 14 are mixed to form a grain mixture 16. The anti-aging ingredients 12 may include vitamin A, vitamin C, vitamin E, collagen, hydrolyzed collagen, elastin, niacin, peptides, copper peptides, hexapeptides, palmitoyl oligopeptide, palmitoyl Tetrapeptide-7, pentapeptides, and hydrolyzed silk. As will be understood by one of ordinary skill in the art, the anti-aging ingredients 12 may include any suitable compounds known to have an anti-aging affect. The synthetic grain 14 may include any suitable polymer material, polybutylene terephthalate (PBT), polyethylene terephthalate (PET), nylon, polyester, a thermoplastic elastomer (TPE), any suitable material for constructing threads of a cosmetic brush head, or combination thereof. As an example, the anti-aging ingredients 12 and the synthetic grain 14 may be mixed at a ratio of 1:100 respectively. For example, the grain mixture may be formed from one or more antioxidants and at least one synthetic grain (e.g., one or more polymer materials).

In process block 104, the grain mixture 16 is then melted. The grain mixture 16 may be melted in a hair forming machine 18. For example, the grain mixture 16 may be melted at a temperature in a range of 240° C.-270° C. The grain mixture 16 may be melted at this temperature range for 10 minutes, in the range of 9-11 minutes, or in the range of 8-12 minutes.

In process block 106, the melted grain mixture 16 is then extruded out of the hair forming machine 18 to form a plurality of threads (filaments) 20. For example, the plurality of filaments 20 may comprise an admixture of the anti-aging ingredients 12 and the synthetic grain 14 described above.

In process block 108, the extruded threads 20 are cooled. The extruded threads 20 may cooled by immediately passing the threads 20 into water after being extruded from the hair forming machine 18. The threads 20 may be cooled in the water for 1 minute or for any suitable duration of time.

In process block 110, the threads 20 are passed through at least one thread stretching machine 30. The thread stretching machine(s) 30 may stretch each thread 20 to add elasticity and durability to the threads 20, as well as dry the threads 20. For example, the thread stretching machine(s) 30 may be configured to stretch the threads 20 to a thickness in the range of 0.07 mm-0.1 mm. As would be understood by one of ordinary skill in the art, any suitable thickness may be achieved.

In process block 112, the threads 20 are ironed. The threads 20 may be ironed by passing the threads 20 through an ironing machine 32 to shape the threads 20 in accordance with the desired characteristics of the resulting cosmetic brush head 80. For example, if a fluffier cosmetic brush head 80 is desired, the ironing machine 32 may add larger waves to the threads 20. If a more dense cosmetic brush head 80 is desired, the ironing machine 32 may add smaller waves or flatten the threads 20 altogether. The ironing machine 32 may be set to a temperature in a range of 245° C.-260° C. to achieve the desired shaping capability. As will be understood by one of ordinary skill in the art, the ironing machine 32 may be set to any suitable temperature for achieving the desired characteristics of the threads 20.

In process block 114, the threads 20 are wound. The threads 20 may be wound by a spinner machine 34 that winds the threads 20 circumferentially around a bobbin 36 to form a collection of threads 40. This winding may be continued until the collection of thread 40 reaches 15 cm in thickness. However, as would be understood by one of ordinary skill in the art, any suitable thickness may be achieved.

In process block 116, the collection of threads 40 is cut off of the bobbin 36, resulting in a bundle of threads 42. The collection of threads 40 may be cut with a hair cutting machine 44. The resulting bundle of threads 42 may, for example, have a length of 1.5 m. However, as would be understood by one of ordinary skill in the art, any suitable length may result. The length of the bundle of threads may be determined by the size of the bobbin 36.

In process block 118, the bundle of threads 42 is secured at one end and hung up, so that all the threads in the bundle are hanging vertically.

In process block 120. the bundle of threads 42 is combed through to untangle and smooth out the threads 20 in the bundle of threads 42.

In process block 122, the hanging bundle of threads 42 is covered with a first wrapping 50 and a second wrapping 52 along a length of the bundle of threads 42. The first wrapping 50 may be of a polyethylene material or any suitable material. The thickness of the first wrapping 50 may be 0.1 mm or any suitable thickness. The bundle of threads 42 may be loosely wrapped with the first wrapping 50 such that the bundle of threads 42 is held together in a columnar shape without significant compression of the bundle of threads 42.

The bundle of threads 42 wrapped in the first wrapping 50 is then covered in the second wrapping 52. The second wrapping 52 may be wrapped more tightly around the bundle of threads 42 than the first wrapping 50. Tightening the bundle of threads 42 with the second wrapping 52 may allow for more accuracy and consistency in subsequent cutting of threads 20 in the bundle of threads 42.

In process block 124, the wrapped bundle of threads 42 is then cut into at least two short bundles of thread 60. The short bundles of thread 60 may be cut to have a length of 10 cm each. However, as one of ordinary skill in the art would understand, the short bundles of thread 60 may be cut to different lengths, depending on the desired cosmetic brush head length. The bundle of threads 42 may be cut with a hair cutting machine 62.

In process block 126, the short bundles of thread 60 are soaked in a solution 64. One end of each short bundle(s) of threads 60 may be soaked in a solution 64 to form tapered tips on a distal end of each thread 20 in the short bundles of thread 60. For example, the tapering of the distal ends of the filaments 20 may be performed by etching. The solution 64 may have any suitable properties for forming tapered tips. For example, the solution 64 may be a basic solution for etching the filaments 20. As an example, one end of each of the small bundles of thread 60 may be set in an oxidized tank containing NaOH solution. The NaOH solution may comprise a 4:6 ratio of NaOH to water and the end of each of the bundles of thread 60 may be soaked for 15 minutes. In this example, the NaOH solution eats away at the end of the threads 20 to form the tapered tip on each thread 20. The length of the tapered tip depends on how much of each thread is exposed to the NaOH solution, so for example, only 1.5 cm of the end of each of the bundles of thread 60 may be soaked in the NaOH solution to form a tapered tip length of 1.5 cm on each thread 20. As one of ordinary skill in the art would understand, other suitable values may be used according to any desired tapered tip length.

In process block 128, the short bundles of thread with tapered tips 66 are removed from the solution 64 and are rinsed. The short bundles of thread with tapered tips 66 may be rinsed with water for 1 minute.

In optional process block 130, where the threads 20 are dyed, the short bundles of thread with tapered tips 66 are rinsed with a dying agent that is configured to dye the threads to a desired color. In this embodiment, the dyed threads may be rinsed again to remove the dying agent in optional process block 132.

In process block 134, the short bundles of thread with tapered tips 66 are submerged and soaked in an anti-aging solution. The anti-aging solution may include the same anti-aging ingredients as in process step 102. For example, the anti-aging solution may include one or more of vitamin A, vitamin C, vitamin E, hydrolyzed collagen, niacin, peptides, and hydrolyzed silk. The anti-aging solution may be a ratio of one part anti-aging ingredients to five parts solvent (e.g., water). After the short bundles of thread with tapered tips 66 have been submerged in the anti-aging solution, a hair softener is added to the anti-aging solution. The hair softener may be added such that the ratio of hair softener to anti-aging solution is 1:20. The short bundles of thread with tapered tips 66 may be soaked in the anti-aging solution with hair softener for 24 hours. However as one of ordinary skill in the art would understand, other suitable times may be appropriate.

In process block 136, the short bundles of thread with tapered tips 66 are dried in a drier 70. The drier may be set to a temperature in the range of 75° C. to 85° C. and the short bundles of thread may be dried for 260 minutes. However, as one of ordinary skill in the art would understand, other suitable times and temperatures may be appropriate.

In process block 138, the short bundles of thread with tapered tips 66 are then cooled. The short bundles of thread with tapered tips 66 may be cooled, e.g., to reduce breakage. The short bundles of thread with tapered tips 66 may be cooled in the drier 70 at a temperature in the range of −0.5° C.-0.5° C. The short bundles of thread with tapered tips 66 may be cooled at this temperature range for 200 minutes. However, as one of ordinary skill in the art would understand, other suitable times may be appropriate.

In process block 140, the short bundles of thread with tapered tips 66 are then unbundled and mixed together. Unbundling the short bundles of thread with tapered tips 66 allows for all of the threads to be randomized, e.g., with the threads from other short bundles of thread with tapered tips 66. The unbundled short threads 74 may be randomized on a spinning plate. As threads from different short bundles of thread with tapered tips may have slightly different diameters, tip lengths, thread shape, or other characteristics, randomizing all of the threads ensures consistency of feel and characteristics of the resulting cosmetic brush heads.

In process block 142, the short threads 74 are sorted so that all the tapered tips are facing the same direction. This may be done by bundling the threads 74 again into small bundles and applying a sifting or combing tool to the bundle. The combing tool may be used to extract all bent, curved, or folded hair from the bundle, while the sifting tool may be used to extract the non-tapered tip ends from the tapered tip ends at one end of the bundle. This sorting step ensures that all the threads are straight and the tapered tips are all oriented in the same direction relative to each other.

In process block 144, the sorted threads 74 are collected to form brush bundles (tufts) 76 of a desired size. As one of ordinary skill in the art would understand, the brush bundle 76 may be made to any suitable thickness.

In process block 146, the brush bundles are then wrapped. For example, the brush bundle may be wrapped loosely in plastic. This loosely wrapped brush bundle may then be placed onto a brush shaping surface, such as a concave bowl, with the tapered tips facing the brush shaping surface. The brush bundle may then be gently tapped so that the threads fall into contact with the brush shaping surface, resulting in a brush bundle of the desired brush shape. The wrapping may then be removed and the filaments may be crimped. For example, a string may be tied around the circumference of the brush bundle at the midpoint of the length of the threads, so as to secure the brush bundle. A ring element may then be secured around the non-tapered tip end of the brush size bundle, below the string. The string may then be cut, resulting in a cosmetic brush head 80.

In process block 148, the cosmetic brush head 80 is affixed to an applicator body (styling tool handle) 82 to form a cosmetic brush (applicator, cosmetic styling tool) 84. A length of the tuft 76 of anti-aging filaments may be between 15% and 20% of a length of the applicator 84. A width at a widest of the tuft 76 of anti-aging filaments may be between 50% and 60% wider than a width at a widest point of the applicator body 82. Alternatively or additionally, a width of the applicator body 82 at a widest point may be between 60% and 70% wider than a width of the applicator body 82 at a narrowest point. Alternatively or additionally, a width of the tuft 76 of anti-aging filaments at a widest point may be between 50% and 60% wider than a width of the tuft 76 of anti-aging filaments at a narrowest point.

A shape of the tuft 76 of anti-aging filaments may be configured to conform to a curve of a face of a user when the tuft of anti-aging filaments is brushed against the face of the user. For example, an angle of a top surface of the tuft 76 of anti-aging filaments located opposite the applicator body 82 may be configured to conform to the curve of the face of the user when the tuft of anti-aging filaments is brushed against the face of the user. An a further example, the shape of the tuft 76 of anti-aging filaments may be configured to conform to a cheek of the face of the user when the tuft of anti-aging filaments is brushed against the cheek of the user.

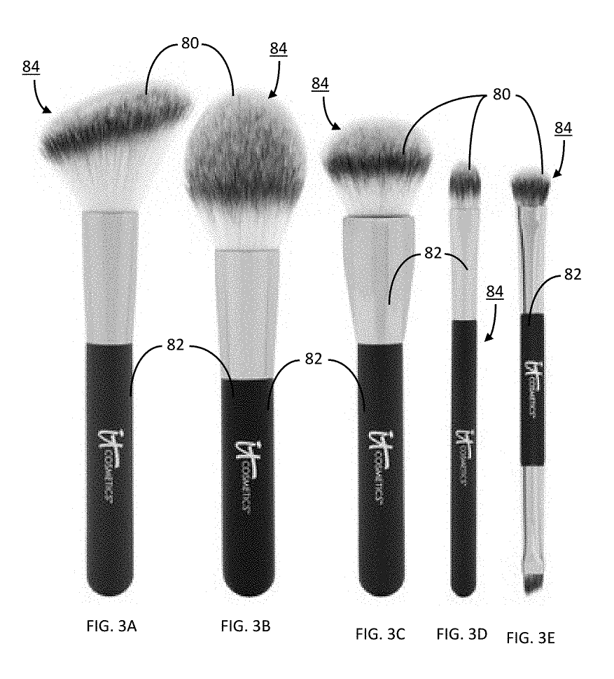

Turning to FIGS. 3A-3E, exemplary embodiments of cosmetic brush heads 80 are shown. The cosmetic brush head 80 may be connected to a cosmetic brush handle 82. As shown, the cosmetic brush head 80 may take various shapes and the cut angle of the brush head 80 may also vary. For example, the brush head 80 may comprise a cross-section having a regular or an irregular geometric shape.

Although the invention has been shown and described with respect to certain embodiments, it is obvious that equivalent alterations and modifications will occur to others skilled in the art upon the reading and understanding of the specification. In particular, with regard to the various functions performed by the above-described components, the terms used to describe such components are intended to correspond, unless otherwise indicated, to any component which performs the specified function of the desired component (e.g., that is functionally equivalent), even though not structurally equivalent to the disclosed component which performs the function of the herein disclosed exemplary embodiment of the invention. In addition, while a particular feature of the invention may have been disclosed with respect to only one embodiment, such feature may be combined with one or more other features as may be desired and advantageous for any given or particular application.

1. An applicator for applying a cosmetic composition, comprising:
a tuft of anti-aging filaments including at least one anti-aging material and at least one of polybutylene terephthalate (PBT), polyethylene terephthalate (PET), nylon, polyester, a thermoplastic elastomer (TPE), or combinations thereof; and
an applicator body physically coupled to the tuft of anti-aging filaments.

2. The applicator for applying a cosmetic composition of claim 1, wherein at least one anti-aging material includes at least one of vitamin A, vitamin C, vitamin E, hydrolyzed collagen, niacin, peptides, antioxidants, and hydrolyzed silk.

3. The applicator for applying a cosmetic composition of claim 1, wherein at least one anti-aging material includes at least one of collagen peptides, elastin, peptides, copper peptides.

4. The applicator for applying a cosmetic composition of claim 1, wherein at least one anti-aging material includes at least one of hexapeptides, palmitoyl oligopeptide, palmitoyl Tetrapeptide-7, and pentapeptides.

5. The applicator for applying a cosmetic composition of claim 1, wherein a length of the tuft of anti-aging filaments comprises between 15% and 20% of a length of the applicator.

6. The applicator for applying a cosmetic composition of claim 1, wherein a width at a widest of the tuft of anti-aging filaments is between 50% and 60% wider than a width at a widest point of the applicator body.

7. The applicator for applying a cosmetic composition of claim 1, wherein a width of the applicator body at a widest point is between 60% and 70% wider than a width of the applicator body at a narrowest point.

8. The applicator for applying a cosmetic composition of claim 1, wherein a width of the tuft of anti-aging filaments at a widest point is between 50% and 60% wider than a width of the tuft of anti-aging filaments at a narrowest point.

9. The applicator for applying a cosmetic composition of claim 1, wherein a shape of the tuft of anti-aging filaments is configured to conform to a curve of a face of a user when the tuft of anti-aging filaments is brushed against the face of the user.

10. The applicator for applying a cosmetic composition of claim 10, wherein an angle of a top surface of the tuft of anti-aging filaments located opposite the applicator body is configured to conform to the curve of the face of the user when the tuft of anti-aging filaments is brushed against the face of the user.

11. The applicator for applying a cosmetic composition of claim 10, wherein the shape of the tuft of anti-aging filaments is configured to conform to a cheek of the face of the user when the tuft of anti-aging filaments is brushed against the cheek of the user.

12. A cosmetic styling tool, comprising:
a brush head having a plurality of filaments including an admixture of one or more anti-aging materials and one or more polymer materials; and
a styling tool handle.

13. The cosmetic styling tool of claim 12, wherein the plurality of filaments comprises an admixture of one or more antioxidants and one or more polymer materials.

14. The cosmetic styling tool of claim 12, wherein the plurality of filaments comprises an admixture of anti-aging material and at least one of polybutylene terephthalate (PBT), polyethylene terephthalate (PET), nylon, polyester, a thermoplastic elastomer (TPE), or combinations thereof.

15. The cosmetic styling tool of claim 12, wherein the plurality of filaments comprises an admixture of one or more of vitamin A, vitamin C, vitamin E, hydrolyzed collagen, niacin, peptides, antioxidants, or hydrolyzed silk, or combinations thereof, and one or more polymer materials.

16. The cosmetic styling tool of claim 12, wherein the plurality of filaments comprises an admixture of one or more of collagen peptides, elastin, peptides, or copper peptides, and one or more polymer materials.

17. The cosmetic styling tool of claim 12, wherein the plurality of filaments comprises an admixture of one or more of hexapeptides, palmitoyl oligopeptide, palmitoyl Tetrapeptide-7, and pentapeptides, and one or more polymer materials.

18. The cosmetic styling tool of claim 12, wherein the brush head comprises a cross-section having a regular or an irregular geometric shape.
